SEQUENCE LISTING (26 sequences)

SEQ ID NO: 1 - protein, 10 aa, artificial sequence
  GnRH II antagonist
XXXSXXXXPX

SEQ ID NO: 2 - protein, 10 aa, artificial sequence
  GnRH II antagonist
XXXSHXWYPX

SEQ ID NO: 3 - protein, 10 aa, artificial sequence
  GnRH II antagonist
XXXSHXWYPX

SEQ ID NO: 4 - protein, 10 aa, artificial Sequence
  GnRH II antagonist
XXXSHXWYPX

SEQ ID NO: 5 - protein, 10 aa, artificial Sequence
  GnRH II antagonist
XXXSHXWYPX

SEQ ID NO: 6 - protein, 10 aa, artificial Sequence
  GnRH II antagonist
XXXSHXWYPX

SEQ ID NO: 7 - protein, 10 aa, artificial Sequence
  GnRH II antagonist
XXXSHXWYPX

SEQ ID NO: 8 - protein, 10 aa, artificial Sequence
  GnRH II antagonist
XXXSYXWYPX

SEQ ID NO: 9 - protein, 10 aa, artificial Sequence
  GnRH II antagonist
XXXSHXWYPX

SEQ ID NO: 10 - protein, 10 aa, artificial Sequence
  GnRH II antagonist
XXXSYXWLPX

SEQ ID NO: 11 - protein, 10 aa, artificial Sequence
  GnRH II antagonist
XXASHXWYPX

SEQ ID NO: 12 - protein, 10 aa, artificial Sequence
  GnRH II antagonist
XXFSHXWYPX

SEQ ID NO: 13 - protein, 10 aa, artificial Sequence
  GnRH II antagonist
XXHSHXWYPX

SEQ ID NO: 14 - protein, 10 aa, artificial Sequence
  GnRH II antagonist
XXXSHXWYPX

SEQ ID NO: 15 - protein, 10 aa, artificial Sequence
  GnRH II antagonist
XRXSHXWYPX

SEQ ID NO: 16 - protein, 10 aa, artificial Sequence
  GnRH II antagonist
XYXSHXWYPX

SEQ ID NO: 17 - protein, 10 aa, artificial Sequence
  GnRH II antagonist
XWXSHXWYPX

SEQ ID NO: 18 - protein, 10 aa, homo sapiens
  MOD_RES (1)..(1): pGlu
EHWSHGWYPG

SEQ ID NO: 19 - protein, 10 aa, artificial sequence
  GnRH II antagonist
XXXSHXWYPX

SEQ ID NO: 20 - protein, 10 aa, artificial sequence
  GnRH II antagonist
XXXSHXWLPX

SEQ ID NO: 21 - protein, 10 aa, artificial sequence
  GnRH II antagonist
XXXSYXWYPX

SEQ ID NO: 22 - protein, 10 aa, artificial sequence
  GnRH II antagonist
XXXSYXWLPX

SEQ ID NO: 23 - protein, 10 aa, artificial sequence
  GnRH II antagonist
XXXSHXWLPX

SEQ ID NO: 24 - protein, 10 aa, artificial sequence
  GnRH II antagonist
XXXSYXWYPX

SEQ ID NO: 25 - protein, 10 aa, artificial sequence
  GnRH II antagonist
XXXSYXWLPX

SEQ ID NO: 26 - protein, 10 aa, artificial sequence
  GnRH II antagonist
XXXSXXXXPX

(19) United States
(12) Patent Application Publication
(10) Pub. No.: US 2012/0004182 A1
(43) Pub. Date: Jan. 5, 2012

(54) PHARMACEUTICAL COMPOSITIONS AND METHODS FOR INDUCTION AND ENHANCEMENT OF APOPTOSIS IN TUMOR CELLS

(76) Inventors: CARSTEN GRUENDKER, Bovenden (DE); GUENTER EMONS, Goettingen (DE)

(21) Appl. No.: 12/829,803

(22) Filed: Jul. 2, 2010

(51) Int. Cl. A61K 38/08; C12N 5/02; A61P 35/00

(52) U.S. Cl. 514/19.4; 514/21.6; 514/19.3; 435/375

(57) ABSTRACT

The present invention relates to methods for inducing and/or enhancing apoptosis in pathogenic cells. In particular, the present invention relates to the use of GnRH II antagonists in combination with at least one further compound selected from the group of selective estrogen receptor modulators (SERM), Aromatase inhibitors, and glycolysis inhibitors or salts or solvates thereof for inducing and/or enhancing apoptosis of specific types of tumor cells, expressing the GnRH II receptor as well as to methods relating thereto.

The present invention relates to methods for inducing and/or enhancing apoptosis in pathogenic cells. In particular, the present invention relates to the use of GnRH II antagonists in combination with at least one further compound selected from the group of selective estrogen receptor modulators (SERM), selective estrogen receptor down regulator (SERD), aromatase inhibitors, and glycolysis inhibitors or salts or solvates thereof for inducing and/or enhancing apoptosis of specific types of tumor cells, expressing the GnRH II receptor as well as to methods relating thereto.

### BACKGROUND

The hypothalamic decapeptide gonadotropin-hormone-releasing hormone (GnRH), also known as lutenizing hormone-releasing-hormone (LHRH), functions as a key hormone in the regulation of mammalian reproduction. It is released from the hypothalamus and stimulates the synthesis and release of lutenizing hormone (LH) and follicle stimulating hormone (FSH). In addition to its classic hypophysiotropic action, GnRH functions as a modulator of the activity of diverse systems in the brain and many peripheral organs. It has been suggested that an autocrine/paracrine function of GnRH exists for example in the placenta, granulosa cells, myometrium and lymphoid cells. In addition, this GnRH based autocrine system seems to be present in a number of human malignant tumors including cancers of the ovary, endometrium, breast and other reproductive organs.

About 80% of the endometrial and ovarian cancers and about 50% of all breast cancers, and a large number of the malignant melanoma have GnRH receptors as part of their negative autocrine regulation system for cell proliferation. These types of cancer cells or tumor cells can also be identified as steroid-related or steroid-sensitive tumor cells. In normal tissue GnRH receptors are not present or only expressed very weakly.

The GnRH is a peptide and, today, various forms of the GnRH have been described. One type of GnRH is the mammalian GnRH (mGnRH or GnRH I), which was first found in the mammalian hypothalamus. The second type of GnRH (GnRH II) was described for the first time in chicken brain. GnRH II can be found in almost all vertebrates including mammalia. Beside the expression in the central nervous system the expression of GnRH-I and GnRH-II has been reported in tissues regulating the immune and reproductive system.

As mentioned above, it is known that the GnRH-I and its receptor represents a part of the negative autocrine regulation system for cell proliferation, of the cell cycle as well as in the anti-apoptosis system. The signalling pathway involved in the autocrine regulation system was studied in detail and it was demonstrated that GnRH-I via the nucleus factor kappa B (NF-κB) protects tumor cells from going into the apoptosis.

It was known that in tumor cells GnRH-I agonist and antagonist act in the same way. i.e. both display an anti-proliferative activity. That means, GnRH-I antagonists act like agonists indicating that the dichotomy of GnRH-I agonist and antagonist does not exist in tumor cells. Further, it is described that the anti-proliferative effect of the GnRH-I agonist can be abrogated in cells wherein the signalling pathway via the GnRH I receptor is interrupted. In contrast, the GnRH-I antagonist still has an anti-proliferative effect on this type of cells.

Agonists and antagonists of the GnRH type II molecules of non human mammalian species are described in the art. For example, WO 00/32218 describes pharmaceutical formulations containing GnRH II and antagonists thereof. In WO 03/093304 various agonists and antagonists of the GnRH II are disclosed useful for the treatment of reproductive physiology diseases and steroid-related cancer cells. It was demonstrated that the anti-proliferative effects of GnRH type II agonists as well as of the native GnRH II is higher than the anti-proliferative effect demonstrated for the GnRH I analoga. Additional Antagonists of GnRH II are described in WO 2007/012430.

It was known that GnRH I and GnRH II as well as GnRH I analoga and GnRH II agonists are able to influence the proliferation of tumor cells. That is these molecules can decrease the proliferation rate of the tumor cells, thereby stopping or reducing the growth of the tumor. However, the ultimate goal in tumor treatment, the full disappearance of the tumor, i.e. the full remission of the tumor, can not be achieved when stopping the proliferation of the tumor cells only. More importantly, it is necessary that the tumor and all pathogenic cells whether present in the solid tumor or present as dissimilated cells, are removed e.g. by killing the tumor cells. Thus, full remission can be achieved. Typically, the removal of pathogenic cells may be obtained by surgery or by the induction of cell death. It is well known that the abnormal inhibition of apoptosis is a hallmark of cancer or other diseases. That is, it is desirable to treat tumor diseases not only by stopping the proliferation of tumor cells but also by inducing cell death of the tumor cells. A possibility to drive cell into the cell death is to induce the cell death program by apoptosis.

For decades, one of the mainstay treatments for breast cancer in humans has been the administration of tamoxifen and to a lesser extent, toremifene. Tamoxifen and toremifene are commonly referred to as Selective Estrogen Receptor Modulators (SERMs). The efficacy of the SERMs is putatively based on the ability to compete with endogenous estrogens (e.g., 17[beta]-estradiol), thereby blocking the proliferative effects of these endogenous estrogens on mammary tissue. Both tamoxifen and toremifene are associated with side effects including hot flushes and stimulation of the endometrium in non-hysterectomized women, leading to an increase in uterine bleeding and uterine cancer. Interestingly, tamoxifen has been shown to confer a positive, estrogen-like benefit on the bone despite having an anti-estrogenic like effect on the breast.

The molecular basis of SERM activity involves binding of the ligand SERM to the estrogen receptor (ER), causing conformational changes which facilitate interactions with coactivator or corepressor proteins, and subsequently initiate or suppress transcription of target genes.

A SERM originally was defined as a compound that binds with high affinity to the ER, without significant binding activity to any other nuclear receptor, and therefore, also applies to the estrogens themselves. In addition, however, a SERM was defined to induce “estrogen agonistic” activities in some tissues, and “estrogen antagonistic” activities in others. Based on recent evidence, this definition can now be further modified, as the interaction between a particular SERM and the ER results in a response in a given tissue which cannot necessarily be characterized simply as either “estrogen agonistic” or “estrogen antagonistic”.

Another approach in tumor therapy includes the administration of selective estrogen receptor down-regulators (SERDs), like Fulvestrant, Robertson, 2001, Br. J. Cancer 85, Suppl. 2, 11-14. These types of estrogen receptor antagonists allow reducing the amount of estrogen receptors, thus, inhibiting the synthesis of estrogen, a property not described for other anti-estrogen molecules. Thus, SERD represent a new class of molecules capable of modulating the level and activity of the estrogen receptor, fulvestrant is a typical representative thereof. These compounds are mechanistically distinct from estrogen receptor ligands such as 4-hydroxytamoxifen, which act either as agonists or antagonists, depending on the target tissue. In contrast, SERD, like fulvestrant, have a more antagonistic profile than SERMs, Kieser et al., J Med Chem, 2010, 53(8), 3320-3329.

More recently, aromatase inhibitors have become popular in the treatment of ER(Estrogen Receptor)-dependent breast cancers. Aromatase inhibitors work by blocking the conversion of precursor compounds (e.g., androstenedione) into estrogens, such as estrone. Popular aromatase inhibitors include both steroidal agents, such as exemestane, and non-steroidal agents, such as letrozole and anastrozole. Despite the growing acceptance of the aromatase inhibitors, they suffer from notable side effects including bone loss, increased bone fractures, vasomotor disturbances (e.g., hot flashes) and joint aches and pains. These effects are what one might expect given the induction of estrogen withdrawal that the agents are precipitating. The combination of tamoxifen and anastrozole was included in the very large ATAC trial (Arimidex Tamoxifen Alone and in Combination trial). The combination arm of the trial (i.e., tamoxifen and anastrozole) was terminated early due to the failure to note any additional benefit relative to the tamoxifen monotherapy arm. By the conclusion of the study, anastrozole by itself appeared to be more effective at preventing breast cancer recurrence than tamoxifen. Despite the efficacy of aromatase inhibitors (e.g. anastrozole) and SERMs, such as tamoxifen and toremifene, both have serious issues for which combination therapy does not provide useful answers. A common method for treating hormone-dependent conditions, such as hormone-dependent breast cancer or hormone-dependent prostate cancer, is to treat a patient with an agent that shuts down the endogenous production of sex hormones (e.g. estradiol and testosterone). These agents include gonadotropin releasing hormone agonists (GNRH agonists), such as buserelin, goserelin, histrelin, leuprorelin, nafarelin and triptorelin as well as gonadotropin releasing hormone antagonists (GNRH antagonists), such as abarelix, cetrorelix and ganirelix.

There is extensive scientific and patent literature on GnRH type I agonists and antagonists, which are used in various ways in cancer treatment. The literature on GnRH-II is considerably less extensive than for GnRH-I. However, there might be a cross-talk between the receptors for GnRH-I and GnRH-II.

Several patent applications relate to the use of GnRH-H antagonists, however, a combination with other anti-cancer agents is not suggested, WO2007012430, WO2007052854.

In US 20080138379, agents are identified useful for developmental deformations and cancer. Among an extensive list of agents proposed for use alone or in combination GnRH and LHRH appear.

Although other GnRH antagonists have been suggested to be used in combination therapies, synergistic effects have not been demonstrated. Anti-estrogenic compounds are proposed for use in combination with GnRH antagonists or agonists in US20090062258.

In view of the above, there is an ongoing need for improved tumor therapies, in particular, on hormone receptor sensitive cancer.

Thus, an object of the present invention is to provide pharmaceutical compositions for inducing or enhancing apoptosis of tumor cells or precursor cells thereof of hormone receptor positive tumors. In another aspect, the present invention relates to methods for inducing or enhancing the apoptosis of tumor cells or precursor cells thereof of gynaecological cancer, like endometrial cancer or ovarian cancer as well as of breast cancer and malignant melanoma. In addition, the present invention aims to provide methods for treating subjects afflicted with said diseases.

### SUMMARY OF THE INVENTION

The present invention is directed, in part, to the novel and unexpected finding that GnRH-II antagonists in concert with a further compound selected from the group of selective estrogen receptor modulators (SERM) selective estrogen receptor down-regulator (SERD), aromatase inhibitors, and glycolysis inhibitors or salts or solvates thereof can induce or enhance apoptosis in tumor cells or precursor cells thereof of specific types of cancers expressing the GnRH-II receptor, and, thus, having an anti tumor effect. It has been recognized that a synergistic, effect is given when combining GnRH-II antagonists with the at least one further compound defined above. Further, it has been recognized that tumors resistant to a compound selected from the group of selective estrogen receptor modulators (SERM), SERD, aromatase inhibitors, and glycolysis inhibitors may regain sensitivity to said therapeutics when using the combination of therapeutics according to the present invention.

Hence, the present invention provides a pharmaceutical composition comprising at least one GnRH II antagonist in combination with at least one further compound selected from the group of selective estrogen receptor modulators (SERM), selective estrogen receptor down-regulator (SERD), aromatase inhibitors, and or glycolysis inhibitors or salts or solvates thereof and, optionally, a pharmaceutically acceptable carrier.

In a preferred embodiment of the present invention, the GnRH II antagonist is at least a compound having the general formula I (Seq. ID No. 1):

X₁-X₂-X₃-Ser-X₄-X₅-X₆-X₇-Pro-X₈-NH₂ (I)

wherein

### X₁=Ac-D-2Nal, Ac-2 Nal, Ac-Δ³Pro

### X₂=His, D-4 Cpa, Arg, Tyr, Trp, D-4Fpa, D-4 Bpa

### X₃=Trp, D-3 Pal, D-2Nal, Ala, Phe, His

### X₄=Tyr, His

### X₅=D-Lys, D-Trp, D-3 Pal, D-2Nal, Gly, D-Cit

### X₆=Trp, Leu, Arg

### X₇=Tyr, Leu, Arg

### X₈=Gly, Ala, D-Ala, D-Gly, D-Cys, D-Ser, D-Val, D-Thr, D-Pro, D-Ile, D-Leu

or derivatives thereof, and at least one further compound selected from the group of selective estrogen receptor modulators, selective estrogen receptor down-regulator, aromatase inhibitors, and or glycolysis inhibitors or salts or solvates thereof and optionally a pharmaceutically acceptable carrier.

Further, the present invention provides pharmaceutical compositions for use in inducing and/or enhancing apoptosis tumor cells or precursor cells thereof, in particular, in steroid dependent tumor cells or precursor cells. The pharmaceutical compositions according to the present invention are particularly useful for the prophylaxis or treatment of breast cancer, gynaecological cancer, prostate cancer or malignant melanoma, in particular, when said tumor cells of the above types of cancer express GnRH-II receptor.

Further, the present invention relates to methods for inducing and/or enhancing apoptosis of tumor cells or precursor cells thereof expressing the GnRH-II receptor comprising the step of administering a first compound of at least one GnRH-II antagonist peptide, e.g. of the general formula J, and at least a second compound selected from the group of SERM, SERD, aromatase inhibitors and/or glycolysis inhibitors.

Finally, the present invention concerns a method for the remission of tumor cells or precursors thereof whereby said tumor cells or precursor cells thereof express the GnRH-II receptor, in particular, for the remission of tumor cells or precursor cells of a gynaecological cancer, prostate cancer or malignant melanoma in a subject comprising the step of contacting the tumor cells or the precursor cells of said type of cancer with at least one GnRH-II antagonist, e.g. of the general formula I, and at least one compound selected from the group of selective estrogen receptor modulators, aromatase inhibitors and/or glycolysis inhibitors.

### BRIEF DESCRIPTION OF THE FIGURES

In FIG. 1A, the results of in vitro administration of GnRH II antagonist alone or in combination with Tamoxifen as a representative of a SERM compound are shown. As demonstrated, the combinatorial administration of GnRH II antagonist with Tamoxifen results in synergistic effects, namely, decreasing the proliferation rate of the cancer cells compared to the administration of the therapeutics alone.

In FIG. 1B the same experiments were repeated using a GnRH II antagonist alone or in combination with Fulvestrant as a representative of SERD, again, a synergistic effect is demonstrated.

In FIG. 2 the results for in vivo treatment with a GnRH II antagonist and Tamoxifen is shown. As demonstrated, the tumor volume decreased over time when administering a combination of GnRH II antagonist and Tamoxifen while the tumour volume, increases administering one of said compounds alone. In addition, a remission of the tumor can be observed when administering a combination of GnRH II antagonist and SERM.

FIG. 3 shows the in vivo results for administering again GnRH II antagonist and Fulvestrant alone or in combination. Again, a synergistic effect is demonstrated showing tumor volume reduction compared to the administration of said compounds alone.

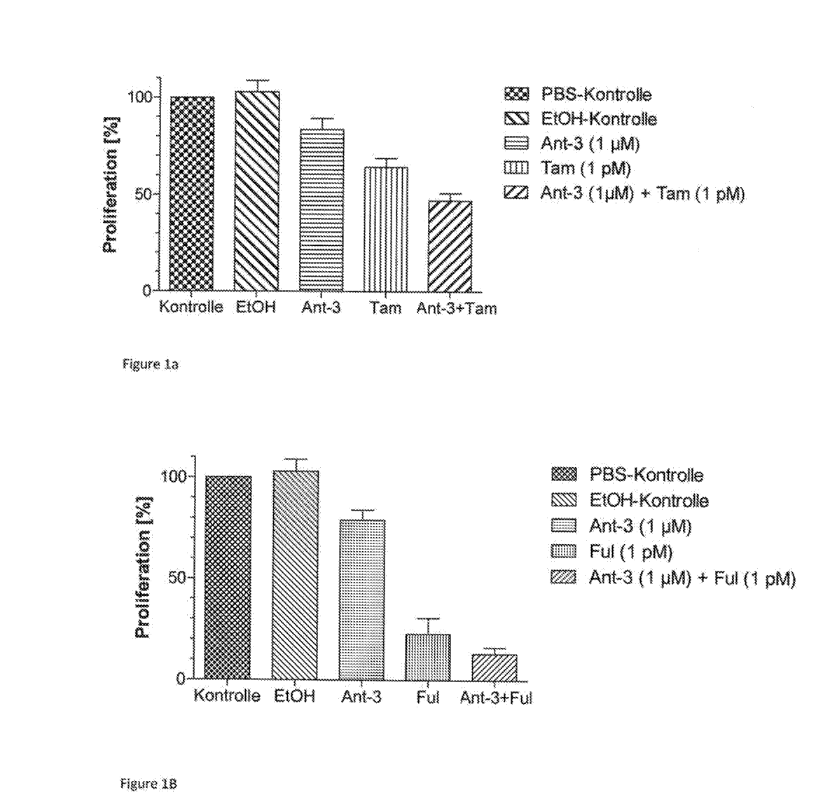

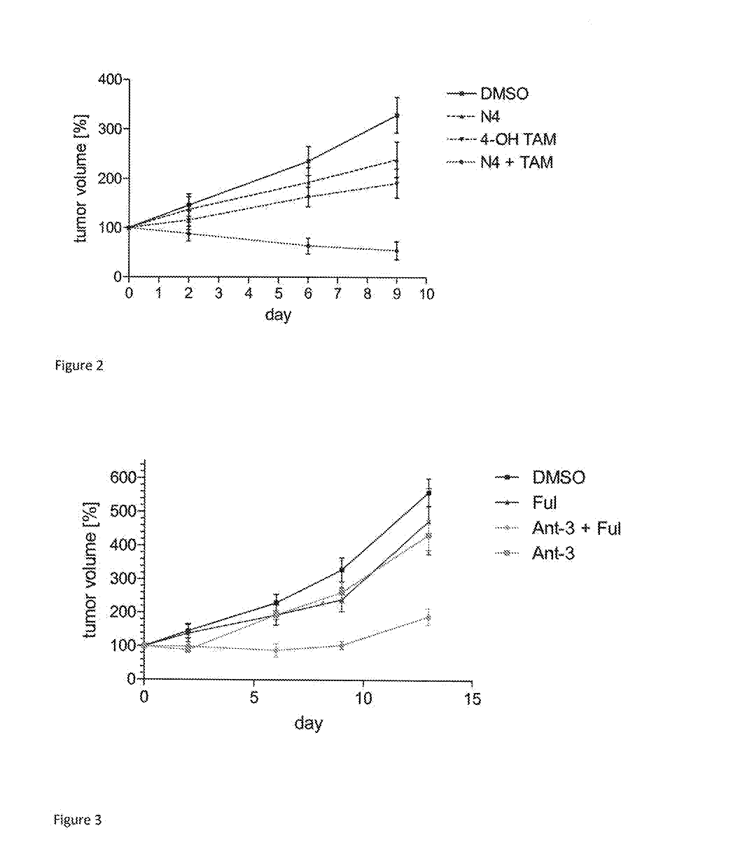

### DETAILED DESCRIPTION OF THE PRESENT INVENTION

The following description of particular embodiments and examples are offered by way of illustration and not by way of limitation. Unless contraindicated or noted otherwise in these descriptions and throughout the specification, the terms “a” and “an” mean one or more, the term “and/or” when occurring herein includes the meaning of “and”, “or” and “all or any other combination of the elements connected by said term”.

The following definitions are set forth to illustrate and define the meaning and scope of the various terms used to describe the invention herein.

“Agonist” refers to a biologically active ligand which binds to its complementary biologically active receptor and activates the latter either to cause a biological response in the receptor or to enhance pre-existing biological activity of the receptor.

“Antagonists” refers to a biologically active ligand which binds to its complementary biologically active receptor and does not activate the latter to cause the natural biological response in the receptor or to reduce pre-existing biological activity of the receptor.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G: Additionally, Ac-(D)-2Nal is Acetyl-β-(2-Naphthyl)-(D)-Alanine; Ac-Δ³Pro is Acetyl-3,4-dehydro-Proline; D-4 Cpa [=(4Cl)-D-Phe] is 4-Chloro-D-Phenylalanine; D-4Fpa [=(4F)-D-Phe] is 4-Fluoro-D-Phenylalanine; D-4 Bpa [=(4Br)-D-Phe] is 4-Bromo-D-Phenylalanine; D-2 Pal is β-(2-Pyridyl)-D-Alanine; D-3 Pal is β-(3-Pyridyl)-D-Alanine; D-2Nal is β-(2-Naphthyl)-D-Alanine; D-Cit is D-Citrulline.

Unless otherwise specifically mentioned, the amino acid residues may be present in its D-form or L-form. Preferred the amino acid residues are in the L-form unless the D-form is specifically identified.

In addition to peptides consisting only of naturally-occurring L- or D-amino acids, peptide mimetics, also known as peptidomimetics or peptide analogs are also provided. Peptide analogs are commonly used in the pharmaceutical industry as non-peptide drugs with properties analogous to those of the template peptide. These types of compound are termed “peptide mimetics” or “peptidomimetics” (Fauchere, J. Adv. Drug Res. 15_{—}29 (1986); Veber and Freidinger TINS p. 392 (1985); and Evans et al. J. Med. Chem. 30_{—}1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as naturally-occurring receptor-binding polypeptide, but have one or more peptide linkages optionally replaced by a linkage selected from the group consisting of: —CH₂NH—, —CH₂S—, —CH₂—CH_—, —CH═CH (cis and trans), —COCH₂—, —CH(OH)CH₂—, and —CH₂SO—, by methods known in the art.

With derivatives of the GnRH II antagonists are meant all kind of peptides and/or proteins and/or fragments thereof, including peptides and/or proteins comprising posttranslational modifications, chemical modifications, enzymatic modifications and modifications due to other mechanisms. In particular, derivatives which do not negatively affect the properties of the GnRH II antagonists as described herein. Derivatives of peptides may comprise amino acid residues different from the standard set of 20 amino acids and/or may comprise peptidomimetic structures. Generally, the term “antagonist(s)” as used herein encompasses also derivatives of said antagonist(s).

The term “gynecological cancers” as used herein refers to cancers derived from tissues of the female reproductive tract such as ovary, fallopian tube, uterus (Endometrium, Myometrium), cervix.

The term “pathogenic cells” as used herein refers to cells which are neoplastic. e.g. tumor cells and precursor cells thereof.

The present inventors surprisingly found that the GnRH II antagonists in combination with the at least one further compound selected from the group of selective estrogen receptor modulators (SERM), selective estrogen receptor down-regulator (SERD), aromatase inhibitors, and glycolysis inhibitors as defined herein not only have anti-proliferative activity, thus, stopping or decelerating tumor growth but also demonstrates an apoptosis inducing activity, thus, driving the tumor cells into the cell death by starting the cell death program and, consequently, allowing remission of the tumor. Consequently, full remission of the tumor may be achievable when applying a the combinatorial GnRH II antagonist treatment according to the present invention.

Generally, a targeted therapy allows to achieve good efficiency with low adverse effects due to its specificity. However, blocking only one signalling pathway is less effective in particular with aggressive tumors. This may be due to the fact that more than one signalling pathways are involved. In growth and proliferation of the tumor cells or, in addition, new signalling pathways are involved which may be due to mutations present in said signalling pathways. Blockage of more than one signalling pathways which may be predetermined in view of their relevance and efficiency, may allow induction of apoptosis in said tumor cells. A potential and specific target in tumor cells are the GnRH receptor molecules.

It was shown in vivo that the efficiency of GnRH analoga with low or medium proliferating and less aggressive tumors is good. However, with aggressive tumor types the efficiency is low although GnRH-II antagonist are more effective than other known therapeutic agents. GnRH-II antagonist inhibit proliferation and growth of endometrial, ovarian and mamma carcinoma cells in vitro and in vivo already in nanomolar concentrations by inducing caspase-3 apoptosis.

Beside GnRH-II antagonist, estrogen receptors are used as specific targets in therapy. In particular, adjuvant therapy in cancer treatment is based on administering selective estrogen receptor modulators (SERM) as well as anti estrogens. Depending on the cell type SERM and anti-estrogens allow the induction of apoptosis or result in cell arrest.

However, these estrogen receptor based therapeutics are less effective with aggressive tumors. Even worse, tumor cells may develop resistance against SERM and anti-estrogen therapeutic agents. A typical example and a widely used representative of SERM is Tamoxifen which induces its tumor activity in mamma carcinoma cells only while the estrogen antagonist faslodex induces apoptosis in mamma, ovarian and endometrial carcinoma cells. However, said anti-tumor effect is remarkably lower with fast growing and more aggressive types of tumors while being effective with low growing and/or medium growing and less aggressive types of tumors.

A combination of targeting at least two different targets is very advantageous. That is, it is demonstrated herein when using a mamma carcinoma cell line MDA-MB-468 as a representative of fast growing and very aggressive tumor cells, a combination of a GnRH-II antagonist with a SERM compound, e.g. 40H-Tamoxifen or Fulvestrant as a representative of a SERD compound, the efficiency in vivo is dramatically increased. That is, the anti-tumor effect of a combination of the GnRH-II antagonist with SERM or SERD have synergistic effects. Moreover, it is possible to achieve remission of the tumor when applying the combinatorial therapy as demonstrated herein. Another advantage of the combination therapy using a GnRH-II antagonist with SERM or SERD or aromatase inhibitor as well as glycolysis inhibitor reside in the possibility to reduce the effective amount of each of the therapeutic compounds administered to the subject and need thereof. Moreover, it may be possible to overcome secondary resistance against one of the compounds used before. For example, secondary resistance against Tamoxifen is described in the art. By combining Tamoxifen or other SERM with the GnRH II antagonist, it is assumed that it is possible to resensities tumor cells which have developed resistance against said SERM before. In this connection, it is noted that the term resistance means that said tumor cells can not be treated at a predetermined concentration of said SERM although at higher concentrations of said compounds, the tumor cells may be killed. However, higher concentrations of said therapeutics may not be possible due to increasing side effects.

Hence, it is possible to reduce side effects known for each of the therapeutics when administered alone while synergistic effects are obtained in particular in view of the treatment of fast growing and more aggressive tumor cells. Not to be bound by theory, it is noted that GnRH-II antagonist induces apoptosis by the GnRH receptor mediated activation of stress activated MAPKs p38/SAPK2 and JNK/SAP1. Activation of p38 as well as of JNK1 is directly associated with induction of apoptosis. p38 and JNK causes translocation of the proapoptotic factors Bax to the mitochondrial membrane and its phosphorisation. By shifting the balance of antiapoptotic signal to proapoptotic signals (e.g. Bax or Bad), mitochondrial membrane is permeabeliesed and cytochrome C is released.

SERMs as well as anti-estrogens are able to induce cell arrest and apoptosis depending on the cell type. When inducing apoptosis, genomic/classic as well as non-genomic signalling pathways are involved. The fast non-genomic effect may be mediated via the membrane estrogen receptor. Activation of p38 and JNK seems to play an important role, thus, leading to activation of the proapoptotic factors Bax.

It is assumed that GPR30, a G protein couple seven transmembrane receptor, plays an important role inducing apoptosis of estrogen sensitive carcinoma cells. The inventors screened available ovarian, endometrial and mamma carcinoma cell lines for GPR30 expression on nucleic acid and protein level showing expression thereof, accordingly.

In a preferred embodiment, the at least one further compound present in the pharmaceutical composition comprising a GnRH II antagonist, like trptorelix, in particular, the GnRH II antagonist of a general formula I is a selective estrogen receptor modulator. Said SERM are well known in the art. Subclasses thereof are: Triphenylethylencs, including Tamoxifen, Toremifene, Droloxifene, Ospemifene, Idoxifene; Chloroethylene, including Clomiphene: Naphtalene, including Lasofoxifene; Benzothiophenes, including Raloxifene, Arzoxifene; Benzopyrans, including Ormeloxifene, Levormeloxifene, Acolbifene (EM-800); Indols, including Bazedoxifene, Pipendoxifene; and steroidals like Fulvestrant, HMR 3339

Particular preferred, the SERM compound is Tamoxifen or Toremifen.

By combining the SERM with the GnRH II antagonist, it is possible to reduce the effective amount of the SERM compound. Hence, adverse effects described for SERM can be reduced as well. Moreover, it is possible to overcome resistance against SERM.

In another preferred embodiment, the further compound is a SERD compound, like Fulvestrant. By combining the GnRH II with the SERD, it is possible to reduce the effective amount of the SERD compound. Hence, adverse effects described for SERD can be reduced as well. Moreover, it is possible to overcome resistance against SERD.

In another preferred embodiment, the at least one further compound is an aromatase inhibitor (AI). Aromatase inhibitors are a class of drugs used in the treatment of breast cancer and ovarian cancer in postmenopausal women. Aromatase is an enzyme that synthesizes estrogen and aromatase inhibitors blocks the synthesis of estrogen, accordingly.

In fact, in postmenopausal women, the main source of estrone and estradiol is the conversion of androgens by aromatase in peripheral tissues. Therefore, inhibition of the aromatase enzyme was proposed as a strategy for treating postmenopausal women with estrogen-dependent breast cancer.

According to Kuerer, H. M, et. al., Journal of Surgical Oncology 2001; 77; 139-147, the aromatase inhibitors are characterised as follows: Aromatase inhibitors can be classified as “suicide inhibitors” (irreversible, type I) and competitive inhibitors (reversible, type II); steroidal and nonsteroidal inhibitors; or non-selective (aminogluthethimide and testolactone) and selective inhibitors (formestaane, anastrozole, fadrozole, letrozole and vorozole).

Subclasses, with typical Al's are:

Steroidal irreversible selective inhibitors, including Formestane (Lentaron) and Exemestane (Aromasin); Nonsteroidal reversible selective inhibitors, including Fadrozole, Anastrozole (Arimidex), Letrozole (Femara), Vorozole (Rivizor); Nonselective Al's, including Aminoglutethimide, Testolactone (Teslac); unclassified Al's, including 4-androstene-3,6,17-trione (6-OXO), 1,4,6-androstatrien-3,17-dione (ATD), 4-hydroxyandrostenedione.

In particular preferred, the aromatase inhibitor is one selected from the group of: Aromasin and Arimidex.

Another preferred embodiment of the present invention relates to a pharmaceutical composition wherein the further compound is a glycolysis inhibitor.

That is, one of the most prominent metabolic alterations in cancer cells is the increase in aerobic glycolysis and the dependency on glycolytic pathway for ATP generation, known as the Warburg effect. The biochemical and molecular mechanisms leading to increasing aerobic glycolysis in cancer cells are rather complex and can be attributed to multiple factors such as mitochondrial dysfunction, hypoxia and oncogenic signals.

Cancer cells consume far more glucose than normal cells to maintain sufficient ATP supply for their active metabolism and proliferation. The metabolic feature has led to the hypothesis that inhibition of glycolysis may severely abolish ATP generation in cancer cells and thus may preferentially kill the malignant cells.

Glycolytic inhibitors and compounds that modulate glycolytic metabolism include among others: 2-Deoxyglucose (inhibits phosphorylation of glucose by hexokinase), Lonidamine (inhibits glycolysis and mitochondrial respiration, inhibits hexokinase, disassociating hexokinase from mitochondria), 3-Bromopyruvate (inhibits hexokinase, acts as an alkylating agent), Imatinib (inhibits Bcr-Abl tyrosine kinase, causes a decrease in hexokinase and G6PG (glucose-6-phosphate dehydrogenase) activity, Oxythiamine/suppresses pentose phosphate pathway by inhibiting transketolase, inhibits pyrovate dehydrogenase). Specific well known glycolysis inhibitors include 6-aminonicotinamide, Genistein, 5-Thioglucose, Mannoheptulose, alpha-clorohydrin, ornidazole, oxalate, pentavalent arsenic compounds.

The glycolytic inhibitors may act on hexokinases, preferred embodiments are 2-Deoxyglucose (glucose analoga), 5-Thioglucose (glucose analoga), Lonidamine (derivative of indazole-3-carboxylic acid), 3-Bromopyruvate, Imatinib (Gleevec).

Another examples of glycolytic inhibitors inhibiting glucokinase are Mannoheptulose (glucose analog). Further the GI include molecules inhibiting Glyceraldehyde 3-phosphate dehydrogenase (GAPDH) like alpha-Chlorohydrin, and Ornidazole (inhibits GAPDH and triosephosphate isomerase).

In addition, the GI is an inhibitor of the pentose phosphate pathway, e.g. of the transketolase, like Oxythiamine (thiamine antagonist; inhibits transketolase and pyruvate dehydrogenase) or an inhibitor of the glucose-6-phosphate dehydrogenase, like 6-aminonicotinamide.

Other examples of GI's include Genistein (natural compound in soybean), oxalate, and pentavalent arsenic compounds.

In a preferred embodiment, the GnRH II antagonist according to SEQ ID No. 1 is a peptide wherein Xₛ is D-Lys, D-3 Pal or D-Trp and/or X₈ is D-Ala.

In another preferred embodiment, the peptide of the general formula I is a peptide wherein X₁ is Ac-D-2Nal, X₂ is D-4-Cpa, D-4-Fpa or D-4 Bpa, X₃ is D-3 Pal, Xₛ is D-Lys or D-3 Pal, X₆ is Trp, X₇ is Tyr or Leu and X_{g} is D-Ala.

Particularly, the peptide is a peptide of SEQ ID Nos. 19, 20 or 23.

In particular the preferred peptide is Seq. ID. No. 23: Ac-D-2Nal-D-4 Cpa-D-3 Pal-Ser-His-D-3 Pal-Trp-Leu-Pro-D-Ala-NH₂.

In particular, the present invention is useful in the treatment of specific types of cancer, like breast cancer and/or malignant melanoma . . . . Furthermore, the present invention is useful in the treatment of gynaecological cancers, particularly of ovarian or endometrial cancer. Furthermore, the present invention is useful in the treatment of prostate cancer.

Thus, the present invention relates to a method of administering GnRH II antagonists or derivatives thereof, in particular, GnRH II antagonists having the sequence of Seq. ID No. 1 in combination with at least one further compound selected from the group of selective estrogen receptor modulators (SERM), selective estrogen receptor down-modulators (SERD), aromatase inhibitors, and or glycolysis inhibitors, to a subject suffering from cancer, in particular of the specific types of cancer as mentioned herein. The GnRH II antagonists in combination with at least one further compound selected from the group of SERM, SERD, aromatase inhibitors, and or glycolysis inhibitors may be administered as pharmaceutical compositions known in the art. The pharmaceutical composition contains at least one GnRH II antagonist or derivatives thereof but also may contain at least two different GnRH II antagonists. The GnRH II antagonists in combination with at least one further compound selected from the group of SERM, SERD, aromatase inhibitors, and or glycolyse inhibitors are particularly useful in cancer therapy, preferably of the specific types of cancer mentioned herein.

Hence, in a further aspect, the present invention relates to pharmaceutical compositions comprising GnRH II antagonist(s) or derivatives thereof and, optionally, a pharmaceutically acceptable carrier.

Preferably, the pharmaceutical composition comprises as GnRH II receptor a compound having the general formula (Seq.-ID No.26):

X₁-X₂-X₃-Ser-X₄-X₅-X₆-X₇-Pro-X₈-NH₂

wherein

### X₁=Ac-D-2Nal, Ac-Δ³Pro

### X₂=His, D-4 Cpa, Arg, Tyr, Trp, D-4Fpa, D-4 Bpa

### X₃=Trp, D-2Nal, Ala, Phe, His

### X₄=Tyr, His

### X₅=D-Lys, D-Trp, D-3 Pal, D-2Nal

### Xₒ=Trp, Leu, Arg

### X₇=Tyr, Leu, Arg

### X₈=D-Ala, D-GIy, D-Cys, D-Ser, D-Val, D-Thr, D-Pro, D-Ile, D-Leu

or derivatives thereof as an active ingredient, optionally together with a pharmaceutically acceptable carrier. Preferably, the active ingredient is a compound according to Seq. ID No. 26 wherein X₁ is Ac-D-2Nal, X₂ is D-4-Cpa, D-4-Fpa or D-4 Bpa, X₃ is D-3 Pal, X₅ is D-Lys or D-3 Pal, X₆ is Trp, X₇ is Tyr or Leu and X₈ is D-Ala. Particular preferred the active ingredient in the pharmaceutical composition is a compound selected from the group of Seq. ID. Nos. 19 to 25.

The pharmaceutical compositions comprise a therapeutically effective amount of the GnRH II antagonist(s) in combination with at least one further compound selected from the group of SERM, SERD, aromatase inhibitors, and or glycolysis inhibitors and, optionally, a pharmaceutically acceptable carrier. The pharmaceutical composition may be administered with a physiologically acceptable carrier to a patient, as described herein. In a specific embodiment, the term “pharmaceutically acceptable” means approved by a regulatory agency or other generally recognized pharmacopoeia for use in animals, and more particularly in humans. The term “carrier” refers to a diluent, adjuvant, excipient, or vehicle with which the therapeutic is administered. Such pharmaceutical carrier's can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice, flour, chalk, silica gel, sodium stearate, glycerol monostearate, talc; sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, patches and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium, carbonate, etc. Examples of suitable pharmaceutical carriers are described in “Remington's Pharmaceutical Sciences” by E. W. Martin (18th ed., Mack Publishing Co., Easton, Pa. (1990)). Such compositions will contain a therapeutically effective amount of the aforementioned GnRH II antagonist or its derivatives, preferably in purified form, together with a suitable amount of carrier so as to provide the form for proper administration to the patient. The formulation should suit the mode of administration.

Typically, pharmaceutically or therapeutically acceptable carrier is a carrier medium which does not interfere with the effectiveness of the biological activity of the active ingredients and which is not toxic to the host or patient.

In another preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in a unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical composition for use in connection with the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

“Therapeutically- or pharmaceutically-effective amount” as applied to the compositions of the instant invention refers to the amount of composition sufficient to induce a desired biological result. That result can be alleviation of the signs, symptoms, or causes of a disease, or any other desired alteration of a biological system. In the present invention, the result will typically involve a decrease in the immunological and/or inflammatory responses to infection or tissue injury and/or decreased tumor growth and/or tumor volume decrease, and/or tumor necrosis, and/or tumor apoptosis.

In vitro assays may optionally be employed to help identifying optimal dosage ranges. The precise dose to be employed in the formulation will also depend on the route of administration, and the seriousness of the disease or disorder, and should be decided according to the judgement of the practitioner and each patient's circumstances. Effective doses may be extrapolated from dose-response curves derived from in vitro or animal model test systems. Preferably, the pharmaceutical composition is administered directly or in combination with an adjuvant. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

In the context of the present invention the term “subject” means an, individual in need of a therapy that can be alleviated or cured by administering the GnRH II antagonist(s) in combination with at least one further compound selected from the group of SERM, SERD, aromatase inhibitors, and or glycolysis inhibitors to the individual. Preferably, the subject is a vertebrate, even more preferred a mammal, particularly preferred a human.

The term “administered” means administration of a therapeutically effective dose of the aforementioned pharmaceutical composition comprising the GnRH II antagonist(s) and the at least one further compound selected from the group of SERM, SERD, aromatase inhibitors, and or glycolysis inhibitors to an individual.

The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents may be administered alone or in combination with other treatments.

The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intrarectally, intraperitoneally, intramuscularly, intrapulmonary, vaginally, rectally, or intraocularly. In some instances, for example, in the treatment of wounds and inflammation, the pharmaceutically effective agent may be directly applied as a solution dry spray.

The attending physician and clinical factors will determine the dosage regimen. A typical dose can be, for example, in the range of 0.001 to 1000 μg; however, doses below or above this exemplary range are envisioned, especially considering the aforementioned factors.

For example, the dosage of the at least one GnRH II antagonist is in the range of from 0.01 to 100, like 0.05 to 50, e.g. 0.1 to 10, 0.2 to 1 mg/kg body weight.

For SERM compounds, the preferred dosage is in the range of from 0.1 to 500 mg per day, like 1 to 100 mg per day, e.g. 5 to 50 mg per day. For example, Tamoxifen is administered in a dosage of 20 mg per day to an adult person. SERD compounds may be administered in the same range as the SERM compounds. For example, fulvestrant is administered in a dosage of 500 mg/application. The aromatase inhibitor may be provided in the range of from 0.01 to 100 mg per day, e.g. 0.1 to 10 mg per day, like 0.5 to 5 mg/day. The glycolysis inhibitor is administered in a dosage similar or identical to the dosage of the aromatase inhibitor.

In Table I preferred GnRH II antagonists are shown. The natural GnRH II peptide is Seq. ID. No. 18 corresponding to database entry Acc. No. o43555. Some of the peptides are disclosed in WO 00/32218 and WO 03/093304, respectively.

| SEQ ID | | | | | | | | | | |
|---|---|---|---|---|---|---|---|---|---|---|
| NO | | | | | | | | | | |
| 18 | pGlu | His | Trp | Ser | His | Gly | Trp | Tyr | Pro | Gly-NH₂ |
| 2 | Ac-D-2Nal | D-4Cpa | D-3Pal | | | D-3Pal | | | | D-Ala-NH₂ |
| 3 | Ac-Δ³Pro | D-4Cpa | D-2Nal | | | D-2Nal | | | | D-Ala-NH₂ |
| 4 | Ac-D-2Nal | D-4Cpa | D-3Pal | | | D-2Nal | | | | |
| 5 | Ac-D-2Nal | D-4Cpa | D-3Pal | | | D-Cit | | | | |
| 6 | Ac-D-2Nal | D-4Cpa | D-3Pal | | | D-Lys | | | | |
| 7 | Ac-D-2Nal | D-4Cpa | D-3Pal | | | D-Trp | | | | |
| 8 | Ac-D-2Nal | D-4Cpa | D-3Pal | | Tyr | D-Cit | | | | D-Ala-NH₂ |
| 9 | Ac-D-2Nal | D-4Cpa | D-3Pal | | | D-Cit | | | | D-Ala-NH₂ |
| 10 | Ac-D-2Nal | D-4Cpa | D-3Pal | | Tyr | D-Cit | | Leu | | D-Ala-NH₂ |
| 11 | Ac-D-2Nal | D-4Cpa | Ala | | | D-Lys | | | | |
| 12 | Ac-D-2Nal | D-4Cpa | Phe | | | D-Lys | | | | |
| 13 | Ac-D-2Nal | D-4Cpa | His | | | D-Lys | | | | |
| 14 | Ac-D-2Nal | (4Cl)-D- | D-3Pal | | | D-Lys | | | | |
| | | Phe | | | | | | | | |
| 15 | Ac-D-2Nal | Arg | D-3Pal | | | D-Lys | | | | |
| 16 | Ac-D-2Nal | Tyr | D-3Pal | | | D-Lys | | | | |
| 17 | Ac-D-2Nal | Trp | D-3Pal | | | D-Lys | | | | |
| 19 | Ac-D-2Nal | D-4Cpa | D-3Pal | | | D-Lys | | | | D-Ala-NH₂ |
| 20 | Ac-D-2Nal | D-4Cpa | D-3Pal | | | D-Lys | | Leu | | D-Ala-NH₂ |
| 21 | Ac-D-2Nal | D-4Cpa | D-3Pal | | Tyr | D-Lys | | | | D-Ala-NH₂ |
| 22 | Ac-D-2Nal | D-4Cpa | D-3Pal | | Tyr | D-Lys | | Leu | | D-Ala-NH₂ |
| 23 | Ac-D-2Nal | D-4Cpa | D-3Pal | | | D-3Pal | | Leu | | D-Ala-NH₂ |
| 24 | Ac-D-2Nal | D-4Cpa | D-3Pal | | Tyr | D-3Pal | | | | D-Ala-NH₂ |
| 25 | Ac-D-2Nal | D-4Cpa | D-3Pal | | Tyr | D-3Pal | | Leu | | D-Ala-NH₂ |

Unless otherwise indicated, the amino acids mentioned in the table either in its naturally occur in L-form or in D-form.

As indicated above, the peptide GnRH II antagonist includes derivatives like peptide mimetics of the subject peptides. A peptide mimetic is a non-naturally occurring analog of a peptide which, because of protective groups at one or both ends of the mimetic, or replacement of one or more peptide bonds with non-peptide bonds, is less susceptible to proteolytic cleavage than the peptide itself. For instance, one or more peptide bonds can be replaced with an alternative type of covalent bond (e.g., a carbon-carbon bond or an acyl bond). Peptide mimetics can also incorporate amino-terminal or carboxyl terminal blocking groups such as t-butyloxycarbonyl, acetyl, alkyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl, thereby rendering the mimetic less susceptible to proteolysis. Non-peptide bonds and carboxyl- or amino-terminal blocking groups can be used singly or in combination to render the mimetic less susceptible to proteolysis than the corresponding peptide. Additionally, substitution of D-amino acids for the normal L-stereoisomer can be effected, e.g. to increase the half-life of the molecule.

Accordingly, the peptide mimetics include peptides having one or more of the following modifications:

peptides wherein one or more of the peptidyl [—C(O)NR—] linkages (bonds) have been replaced by a non-peptidyl linkage such as a —CH₂-carbamate linkage [—CH₂—OC(O)NR—]; a phosphonate linkage; a —CH₂-sulfonamide [—CH₂—S(O)₂NR-] linkage; a urea [—NHC(O)NH—] linkage; a —CH₂-secondary amine linkage; or an alkylated peptidyl linkage [—C(O)NR⁶]— where R⁶ is lower alkyl;
peptides wherein the N-terminus is derivatized to a —NRR¹ group; to a —NRC(O)R group; to a —NRC(O)OR group; to a —NRS(O)₂R group; to a —NHC(O)NHR group, where R and R¹ are hydrogen or lower alkyl with the proviso that R and R¹ are not both hydrogen: to a succinimide group; to benzyloxycarbonyl-NH—(CBZ—NH—) group; or to a benzyloxycarbonyl-NE-group having from 1 to 3 substituents on the phenyl ring selected from the group consisting of lower alkyl, lower alkoxy, chloro, and bromo; or
peptide wherein the C terminus is derivatized to —C(O)R² where R² is selected from the group consisting of C1-C6 alkoxy, and —NR³R⁴ where R³ and R⁴ are independently selected from the group consisting of hydrogen and C1-C6 alkyl.

Preferred mimetics have from zero to all of the —C(O)NH— linkages of the peptide replaced by a linkage selected from the group consisting of a —CR₂OC(O)NR— linkages; a phosphate linkage; a —CH₂S(O)₂NR— linkage; a —CH₂NR— linkage; and a —C(O)NR⁶— linkage, and a —NHC(O)NH-linkage where R is hydrogen or lower alkyl and R⁶ is lower alkyl, and wherein the N-terminus of the mimetic is selected from the group consisting of a —NRR¹ group; a —NRC(O)R group; a —NRC(O)OR group; a —NRS(O)₂R group; a —NHC(O)NHR group; a succinimide group; a benzyloxycarbonyl-NH-group; and a benzyloxycarbonyl-NH-group having from 1 to 3 substituents on the phenyl ring selected from the group consisting of lower alkyl, lower alkoxy, chloro, and bromo, where R and R¹ are independently selected from the group consisting of hydrogen and lower alkyl, and still further wherein the C-terminus of the mimetic has the formula —C(O)R² where R² is selected from the group consisting of hydroxyl, lower alkoxy, and —NR³R⁴ where R³ and R⁴ are independently selected from the group consisting of hydrogen and lower alkyl and where the nitrogen atom of the —NR³R⁴ group can optionally be the amine group of the N-terminus of the peptide so as to form a cyclic peptide, and physiologically acceptable salts thereof.

Particularly preferred, the peptides are modified with an acetyl group at the N-terminus. In another embodiment, the peptides are particularly modified with a —NR³R⁴ group wherein each of R³ and R⁴ are hydrogen at the C-terminus.

The term “lower” as used herein, means a C1-C6 group, which may be in a linear, branched or cyclic form.

The present invention is further described by reference to the following non-limiting figures and examples.

### EXAMPLES

### Example 1

### Apoptosis Assays

To quantify apoptosis a procedure similar to that described by Nicoletti et. al. (J. Immunol. Methods 1991; 139:271-279) that was based on detecting advanced DNA degradation has been used. Briefly, a pellet containing 1×10⁶ cells was gently resuspended in 500 mL of hypotonic fluorochrome solution containing 0.1% Triton X-100 (Sigma, Deisenhofen. Germany), 0.1% sodium citrate, and 50 mg/mL propidium iodide (Sigma). The cell suspensions were placed at 4° C. in the dark overnight before flow cytometry analysis of cellular DNA content on a FACScalibur equipment (Becton Dickinson Immunocytometry Systems, Mountain View, Calif.) was performed with Cellquest software (Becton Dickinson Immunocytometry Systems). Cells were exposed to increasing concentrations of the GnRH-II antagonists from 10-13 M to 10-7 M for 72 hours before they were harvested.

### Proliferation Assay

Five hundred cells per well were plated into 96-wll plates (Falcon, Heidelberg, Germany) in 100 μl DMEM/5. % fetal calf serum (FCS, Biochrom Berlin, Germany) without phenol red, with 2 mM glutamine, 50 U/ml penicillin/streptomycin, 2.5 μg/ml amphotericin B, and 1:100 non-essential amino acids. After cell attachment, 100 μl medium or 100 μl GnRH II antagonist solution at increasing final concentrations of 10⁻¹¹ to 10⁻⁵ M was added to the wells in six replicates and incubated for 1 to 5 days at 37° C. and 5% CO₂. Cell number were determined by a calorimetric assay using Alamar Blue (Biosource, Solingen, Germany). The optical density (OD) of the reduced dye is assessed at 570 nm vs. 630 nm after 4 h at 37° C.

### Example 2

### In Vivo Studies in Mice

### Experimental

Female athymic (nude) mice (CD1 nu/nu), 6 to 8 weeks old on arrival, were obtained from Charles River (Sulzfeld, Germany). The mice were housed in sterile cages in a temperature-controlled room with 12-hour light/12-hour dark schedule and were fed autoclaved chow and water ad libitum. All experiments were done according to the German ethical guidelines and the German laws for protection of animals.

Tumors were initiated by subcutaneous injection of 1×10⁷ cancer cells into the right flank. After 2 weeks all animals had developed solid tumors of about 200 mm³ and treatment was initiated. The in vivo experiments were done as follows: 25 nmol of GnRH-II antagonist of Seq. ID. No. 23 without or with 1 μmol of 40H-Tamoxifen per mouse (5 mice per group and control group) were injected intraperitoneally. Treatment was repeated every 2 days. Tumor volumes were measured. The mice were killed after 10 days of treatment. The following cancer cells were used: MCF-7, MDA-MB-231, MDA-MB-435, MDA-MB-453, MDA-MB-468, T-47-D: all breast cancer cell lines; Ovcar-3 and EFO-21 (ovarian cancer), Ishikawa (endometrial cancer cell line), and Hec 1B (endometrial cancer cell line)

The proliferation and induction of apoptosis were analyzed after treatment without or with a GnRH-II antagonist without or with co-treatment with 4OH-Tamoxifen. Nude mice bearing human breast tumors s.c. were treated without or with a GnRH-II antagonist alone or in combination with 4OH-Tamoxifen and the tumor volumes were measured.

As demonstrated herein, the combination of the GnRH-II antagonist with 4OH-Tamoxifen led to a significant reduction of proliferation as well as to a significant increase of apoptosis in comparison with the single-agent treatment. This additive effect could be confirmed in vivo.

Hence, the antitumor effect in vitro and in vivo is strengthened significantly by the combination of the GnRH-II antagonist with 4OH-Tamoxifen. This co-treatment still has another advantage. GnRH analogs can resensitize resistant human breast cancer cells to 4OH-Tamoxifen. GnRH-II antagonists in combination with 4OH-Tamoxifen are suitable drugs for an efficacious and less toxic endocrine therapy for high aggressive breast cancers.

1. A pharmaceutical composition comprising at least a GnRH II antagonist and at least one compound selected from the group of selective estrogen receptor modulators (SERM), selective estrogen receptor down-regulator (SERD), aromatase inhibitors, and or glycolyse inhibitors or salts or solvates thereof and, optionally a pharmaceutically acceptable carrier.

2. A pharmaceutical composition according to claim 1 wherein said GnRH II antagonist has the general formula I (Seq. ID No. 1):
X₁-X₂-X₃-Ser-X₄-X₅-X₆-X₇-Pro-X₈-NH₂ (I)
wherein
X₁=Ac-D-2Nal, Ac-2 Nal. Ac-Δ³Pro
X₂=His, D-4 Cpa, Arg, Tyr, Trp, D-4Fpa, D-4 Bpa
X₃=Trp, D-3 Pal, D-2Nal, Ala, Phe, His
X₃=Tyr, His
X₅=D-Lys, D-Trp, D-3 Pal, D-2Nal, Gly, D-Cit
X₆=Trp, Leu, Arg
X₇=Tyr, Leu, Arg
X₈=Gly, Ala, D-Ala, D-Gly, D-Cys, D-Ser, D-Val, D-Thr, D-Pro, D-Ile, D-Leu
or derivatives thereof.

3. The pharmaceutical composition according to claim 2 wherein X₅ is D-Lys, D-3 Pal or D-Trp and/or X₈ is D-Ala.

4. The pharmaceutical composition according to claim 2 wherein in the general formula I X₁ is Ac-D-2Nal, X₂ is D-4-Cpa, D-4-Fpa or D-4 Bpa, X₃ is D-3 Pal, X₅ is D-Lys or D-3 Pal, X₆ is Trp, X₇ is Tyr or Leu and X₈ is D-Ala.

5. The pharmaceutical composition according to claim 2, wherein the general formula I is selected from the group consisting of Seq. ID Nos. 2 to 25.

6. The pharmaceutical composition according to claim 1 wherein the at least one compound is at least a SERM, and wherein said SERM is selected from the groups consisting of triphenylethylene derivatives, chloroethylene derivatives, naphthalene derivatives, benzothiophene derivatives, benzopyrane derivatives, indol derivatives and steroidal derivatives.

7. The pharmaceutical composition of claim 6 wherein said SERM is selected from the group consisting of Tamoxifen, Toremifene, Droloxifene, Ospemifene, Idoxifene, Clomiphene, Lasofoxifene, Raloxifene, Arzoxifene, Ormeloxifene, Levormeloxifene, Acolbifene, Bazedoxifene, and Pipendoxifene.

8. The pharmaceutical composition according to claim 1 wherein the at least one compound is at least a SERD.

9. The pharmaceutical composition of claim 8 wherein said SERD is fulvestrant.

10. A pharmaceutical composition according to claim 1 wherein the at least one compound is an aromatase inhibitor.

11. The pharmaceutical composition of claim 10 wherein said aromatase inhibitor is selected from formestane, exemestane, fadrozole, anastrozole, letrozole, vorozole, testolactone. 4-androstene-3,6,17-triune. 1,4,6-androstatrien-3,17-dione, hydroxyandrostenedione, and aminogluthethimide.

12. A pharmaceutical composition according to claim 1, wherein the at least one compound is a glycolysis inhibitor.

13. The pharmaceutical composition of claim 12 wherein said glycolysis inhibitor is selected from the group consisting of 2-deoxy-glucose, lonidamine, 3-bromopyruvate, imatinib, and oxythiamine.

14. A method for inducing and/or enhancing apoptosis of tumor cells or precursor cells thereof expressing the GnRH II receptor comprising the step of providing said tumor cells or precursor cells thereof with a GnRH II antagonist and at least one compound selected from the group consisting of selective estrogen receptor modulators, selective estrogen receptor down-regulator, aromatase inhibitor and glycolytic inhibitor.

15. The method according to claim 14 wherein said tumor cells or precursor cells expressing the GnRH II receptor are cells selected from the group consisting of breast cancer, malignant melanoma, gynaecological cancer, and prostate cancer.

16. The method according to claim 14 wherein the tumor cells or precursor cells are cells selected from ovarian or endometrial cancer.

17. The method of claim 14 wherein said GnRH II antagonist has the general formula (Seq. ID No. 1):
X₁-X₂-X₃-Ser-X₄-X₅-X₆-X₇-Pro-X₈-NH₂ (I)
wherein
X₁=Ac-D-2Nal, Ac-2 Nal, Ac-Δ³Pro
X₂=His, D-4 Cpa, Arg, Tyr, Trp, D-4Fpa, D-4 Bpa
X₃=Trp. D-3 Pal, D-2Nal, Ala, Phe, His
X₄=Tyr, His
X₅=D-Lys, D-Trp, D-3 Pal, D-2Nal, Gly, D-Cit
X₆=Trp, Leu, Arg
X₇=Tyr, Leu, Arg
X₈=Gly, Ala, D-Ala, D-Gly, D-Cys, D-Ser, D-Val, D-Thr, D-Pro, D-Ile, D-Leu
or derivatives thereof.

18. The method according claim 14 wherein the at least one compound is selected from the group of Tamoxifen, Toremifene, Fulvestrant, Aromasin, and Arimidex.

19. A method for the remission of tumor cells or precursors thereof of a gynaecological cancer in a subject comprising the step of contacting the tumor cells or the precursor cells of said gynaecological cancer with a GnRH II antagonist and at least one compound selected from the group consisting of selective estrogen receptor modulators, selective estrogen receptor down-regulator, aromatase inhibitor and glycolysis inhibitor.

20. The method of claim 19 wherein said tumor cells or precursors thereof are from a hormone receptor positive type of cancer.

21. The method of claim 20 wherein said hormone receptor positive type of cancer is selected from the group consisting of breast cancer, endometrial cancer or ovarian cancer, and malignant melanoma.

22. The method of claim 19 wherein said GnRH II antagonist has the general formula (Seq. ID No. 1):
X₁-X₂-X₃-Ser-X₄-X₅-X₆-X₇-Pro-X₈-NH₂ (1)
wherein
X₁=Ac-D-2Nal, Ac-2 Nal, Ac-Δ³Pro
X₂=His, D-4 Cpa, Arg, Tyr, Trp, D-4Fpa, D-4 Bpa
X₃=Trp. D-3 Pal. D-2Nal, Ala, Phe, His
X₃=Tyr, His
X₅=D-Lys, D-Trp, D-3 Pal, D-2Nal, Gly, D-Cit
X₆=Trp. Leu, Arg
X₇=Tyr, Leu, Arg
X₈=Gly, Ala, D-Ala, D-Gly, D-Cys, D-Ser, D-Val, D-Thr, D-Pro, D-Ile, D-Leu or derivatives thereof.

23. The method of claim 22 wherein the at least one compound is selected from the group consisting of Tamoxifen, Toremifene, Fulvestrant, Aromasin, and Arimidex.
